(19) United States
(12) Patent Application Publication
(10) Pub. No.: US 2017/0370083 A1
(43) Pub. Date: Dec. 28, 2017

(54) APPARATUS AND METHOD FOR TREATING AND PREVENTING ODORS

(71) Applicant: Jonathan G. Foss, Waconia, MN (US)

(72) Inventor: Jonathan G. Foss, Waconia, MN (US)

(21) Appl. No.: 15/629,626

(22) Filed: Jun. 21, 2017

Related U.S. Application Data
(60) Provisional application No. 62/326,133, filed on Apr. 22, 2016.

(51) Int. Cl. E03D 9/00; A61L 2/22; E03D 9/03; A61L 2/10; A61L 9/14; E03D 9/02; A47K 13/24

(52) U.S. Cl. CPC E03D 9/007; A61L 2/10; A61L 2/22; A61L 9/14; E03D 9/032; E03D 9/037; A61L 2202/25; A47K 13/24; E03D 2009/024; E03D 2009/026; E03D 2009/028; A61L 2209/135

(57) ABSTRACT

This disclosure relates to new methods and compositions for dispensing multiple compositions into a plurality of containers or atmospheres. In one embodiment, this disclosure pertains to treating and preventing offensive odors arising from toilets.

### CROSS REFERENCE TO RELATED APPLICATIONS

This application claims priority to U.S. Application Ser. No. 62/326,133, entitled “APPARATUS AND METHOD FOR TREATING AND PREVENTING ODORS,” filed Apr. 22, 2016, which is incorporated herein in its entirety.

### TECHNICAL FIELD

This disclosure relates to the sanitization and cleaning industries. In particular, this disclosure relates to an apparatus and methods for cleaning and sanitizing bathrooms and toilets.

### BACKGROUND

On average, people eliminate fecal waste between 1 to 3 times per day. Usually people eliminate their fecal waste into a toilet or similar waste disposal system. This fecal elimination process often brings about unwanted odors on account of metabolic byproducts that are contained in feces and/or accompany the evacuation process. These odors originate from the toilet area, where the fecal evaluation takes place and the waste is collected for subsequent disposal. Left untreated, the waste generates odors, which diffuse into the air and adjacent areas.

Popular remedies for toilet related odors include air circulation systems and air fresheners. Air circulation systems, such as fans, replace the contaminated air with fresh air. Air fresheners typically introduce pleasant fragrances to mask unwanted toilet odors. Air fresheners are available in many forms, such as sprays, heated oils, solids, and gels.

Despite existing products for treating bathroom air, the state of art suffers from several limitations. For example, sprays that are used in bathrooms typically treat only the air around the toilet, i.e., the ambient atmosphere. Such sprays do not target the source of the problem, which is the fecal waste in the toilet and odorous volatile molecules emanating from that waste. Most sprays only cover the odor by dispersing fragrances into the area with the unwanted odor. Such air fresheners do not treat the source of the odor itself.

No existing systems or methods treat both the source of the odor problem and also resulting odors. Accordingly, there exists a need to target both the source of the odor and the ambient atmosphere affected by the odor. In particular, there exists a need to treat both the odor source and the affected surrounding air with compositions targeting both the source of the odor problem and the unwanted odors themselves. Accordingly, there exists a need for systems, methods, and devices, for deploying multiple targeted compositions to multiple different places in and around a toilet.

### DETAILED DESCRIPTION

Disclosed herein is a new apparatus for dispensing chemical compositions, for example dispensing multiple compositions into two or more spaces. Disclosed herein is a new apparatus for eliminating bathroom odors. Also disclosed herein are new methods for eliminating bathroom odors.

Disclosed herein is a new apparatus. Also disclosed herein is a new device, which may be used independently or in concert with the disclosed apparatus. Accordingly, in one embodiment, the apparatus disclosed herein includes the device disclosed herein.

Disclosed here is a new apparatus comprising:
a First Container;
a Second Container;
a Third Container;
a First Composition;
a Second Composition;
a First Dispenser;
a Second Dispenser;
a surrounding ambient atmosphere;
wherein the First Dispenser expels the First Composition from within the First Container into the Third Container;
wherein the Second Dispenser expels the Second Composition from within the Second Container into the ambient atmosphere.

In one embodiment, the Third Container comprises water and air. In one embodiment, the Third Container is open to the ambient atmosphere. In one embodiment, the First Composition is held within the First Container and the Second Composition is held within the Second Container.

As used herein, the term “First Container” means a storage device that can be either left open to the atmosphere or closed. In one example, the First Container is a can, bag, bladder, or bottle.

As used herein, the term “Second Container” means a storage device. Within the context of this disclosure the Second Container can be left open to the atmosphere or closed. In one example, the Second Container is a can, bag, bladder, or bottle.

As used herein, the term “Third Container” means a storage device that can be either left open to the atmosphere or closed. In one example, the Third Container is a can, bag, bladder, or bottle. In one example, the Third Container is a tank, chamber, or bowl, such as a toilet or urinal.

As used herein, the term “First Composition” means a chemical or chemical mixture. In one example, the First Composition is an oil and natural oil. In one example, the First Composition is a perfume. In another example, the First Composition is a surfactant. In another example, the First Composition is an oil. In one example, the First Composition is an alcohol. In one example, the First Composition is an antiseptic.

As used herein, the term “Second Composition” means a chemical or chemical mixture. In one example, the Second Composition is an odor absorber. In one example, the Second Composition is a soap. In one example, the Second Composition is a natural oil. In one example, the Second Composition is a perfume. In one example, the Second Composition is an alcohol. In one example, the Second Composition is an antiseptic.

As used herein, the term “First Dispenser” means a device through which a substance can be delivered. In one example, the First Dispenser is a pump system. In one example, the First Dispenser is a nozzle system.

As used herein, the term “Dispenser” includes pressurized or unpressurized delivery systems, such as pressurized spray systems, or gravity mediated drip systems.

As used herein, the term “Second Dispenser” means a device through which a substance can be delivered. In one example, the Second Dispenser is a pump system. In one example, the Second Dispenser is a nozzle system. In one example, the Second Dispenser is an atomizer.

As used herein, the term “ambient atmosphere” means the surrounding atmosphere in which the device or apparatus resides. In one example, the ambient atmosphere is the air within a bathroom or vestibule, which contains a toilet.

As used herein, the term “expels” means to ejecting or causing a material to move out of the container from which it is expelled. In one example, the First Dispenser expels the First Composition from the First Container.

In one embodiment, the First Composition is held within the First Container and the Second Composition is held within the First Container. In one embodiment, at least one of the First Container and the Second Container is a bag on valve container.

As used herein, the term “bag on valve” refers to a pressurized container with a filled bag inside. In one example, the bag inside is a welded bag. In one example, the bag is filled with a composition on the inside and subjected pressure of greater than one atmosphere on the outside. In one embodiment, the bag is contained within a rigid container and a gas having a pressure of greater than one atmosphere fills the space between the bag and rigid container. In one example, the gas creates a pressure on the bag, which expels the composition from the bag.

In one embodiment, at least one of the First Container and the Second Container is a pump bottle.

As used herein, the term “pump bottle” means a bottle with a dispensing mechanism inserted therein. For example, a “pump bottle,” within the context of this disclosure includes conventional trigger spray bottles which can squirt, spray or mist fluids. In some embodiments, the term “pump bottle” includes a bottle with a nozzle, said bottle having a dip tube inserted therein and a means for pumping liquid from within the bottle, up through the dip tube, and out of the nozzle.

In one example, the First Dispenser is attached to the First Container.

In one embodiment of the disclosed apparatus, the First Composition includes one or more ingredients chosen from an oil, a natural oil, a perfume, an oxidant, a soap, an acid, a base, a surfactant, an odor absorber, a cyclodextrin, and an antioxidant.

As used herein the term “oil” refers to a nonpolar liquid that can be viscous at ambient temperatures. Within the context of this disclosure, an “oil” can be characterized as being both hydrophobic and lipophilic, and having high carbon and hydrogen content. In one embodiment, the term “oil” refers to a substance that is substantially immiscible with water, creating distinct layers of material as opposed to a single solution.

As used herein, the term “natural oil” refers to oils containing compounds which may be found in plants or animals as opposed to oils which exist only through man-made synthesis. Examples of natural oils within the context of this disclosure include vegetable oils with ethereal salts of glycerin, organic acids such as stearic acid, oleic acid, and palmitic acid forming stearin, olein and palmitin, respectively.

As used herein the term “perfume” refers to one or more fragrant volatile molecules, such as aromatic compounds.

As used herein the term “oxidant” can refer to a chemical species that can either remove an electron or transfer electronegative atoms to a substrate. An oxidant can also be referred to as an oxidizing agent. Within the context of this disclosure, examples of oxidants include, chlorinating agents, halogenating agents, peroxides, nitrates, oxygen, ozone, and hypohalite.

As used herein the term “soap” refers to a salt of a fatty acid often characterized for being able to allow insoluble particles to be soluble in water. In one example, the term soap refers to sodium stearate with the following structure:

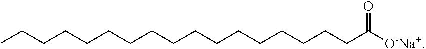

As used herein the term “acid” refers to a chemical substance with a pH of less than 7. In one example, an acid has the ability to turn blue litmus red. In one example, an acid has the ability to react with bases and certain metals to form salts. Within the context of this disclosure, examples of acids include acetic acid, citric acid, phthalic add, sodium hydroxymethylglycinate, hydrochloric acid, and formic acid.

As used herein the term “surfactant” refers to compounds that lower the surface tension (or interfacial tension) between two liquids or between a liquid and a solid.

As used herein the term “odor absorber” refers to a substance, such as a solid or gel, that neutralizes smells. Within the context of this disclosure, one example of an odor absorber is a cyclodextrin.

As used herein the term “cyclodextrin” refers to a family of compounds made up of sugar molecules bound together in a ring. Some examples, but not limited to, are α (alpha)-cyclodextrin (6-membered sugar ring molecule), β (beta)-cyclodextrin (7-membered sugar ring molecule), and γ (gamma)-cyclodextrin (8-membered sugar ring molecule).

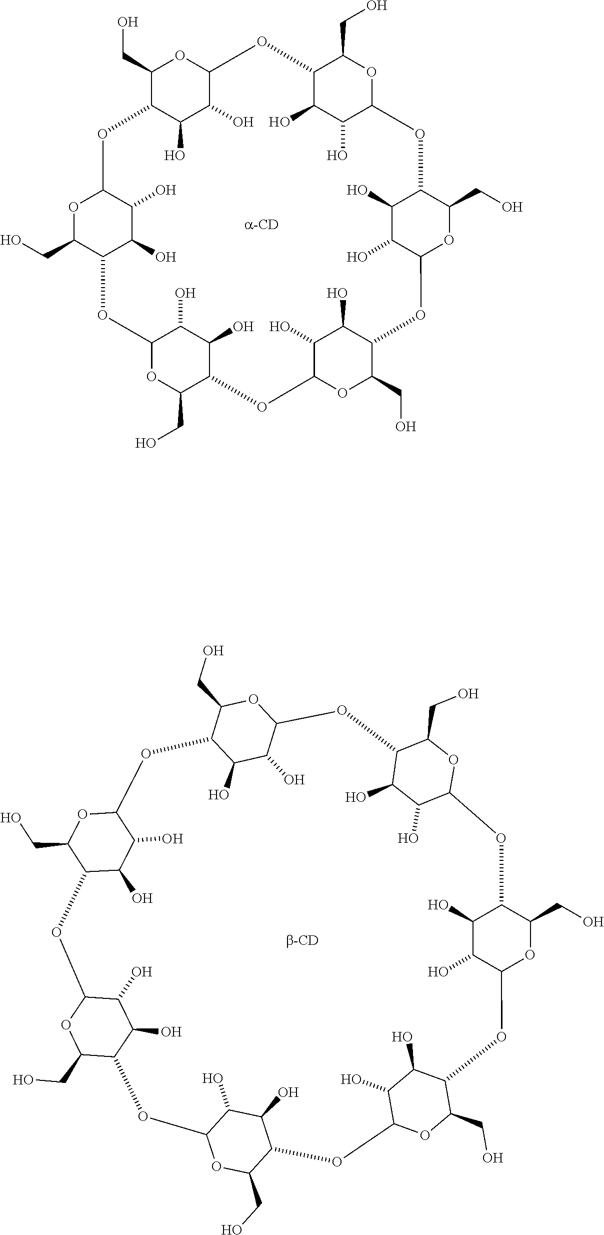

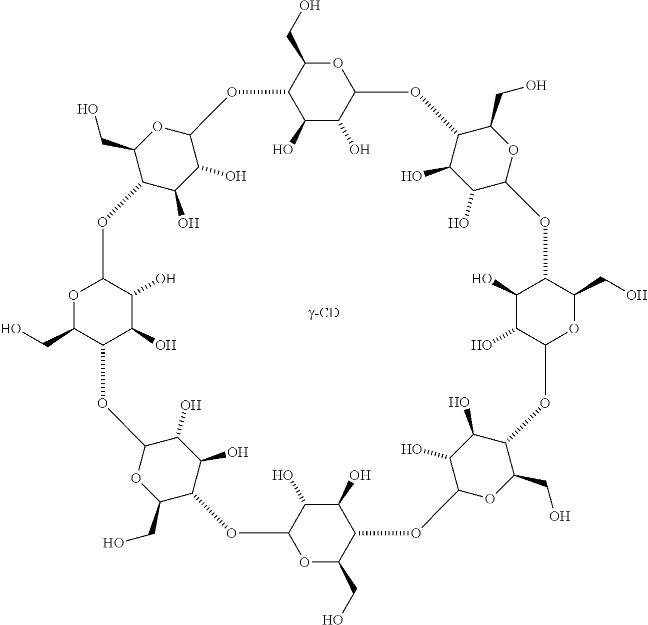

As used herein the term “antioxidant” refers to a chemical agent that lessens oxidation. Within the context of this disclosure, examples of antioxidants include beta carotene, lutein, lycopene, Vitamin C, thiosulfate, Vitamin A, Vitamin E, etc., including salts and derivatives thereof in any proportion.

In one embodiment of disclosed apparatus, the Second Composition includes one or more ingredients chosen from an oil, a natural oil, a perfume, an oxidant, a soap, an acid, a base, a surfactant, an odor absorber, a cyclodextrin, and an antioxidant.

Disclosed herein is a new device comprising:
a Rigid Pressurized Container comprising a First Bladder and Second Bladder; an Outlet;
wherein said First Bladder comprises a composition having a First Bladder Vapor Pressure;
wherein said Second Bladder comprises a composition having a Second Bladder Vapor Pressure;
wherein said Pressurized Container comprises a vapor pressure greater than one atmosphere of pressure; and
wherein said Pressurized Container comprises a vapor pressure greater than said First Bladder Vapor Pressure.

As used herein the term “Rigid Pressurized Container” refers to a container that is sturdy enough withstand pressures higher than 1 atmosphere without compromising the structure of the container. One example of a “Rigid Pressurized Container” is a can or bottle. In one embodiment, the Rigid Pressurized Container is made of plastic. In one embodiment, the Rigid Pressurized Container is made of metal.

As used herein the term “Bladder” mean an inflatable flexible container (e.g., a bag) made of a material capable of holding a liquid composition and capable of bending when exposed to a force, such as a pressure differential. In one example, the bladder is made of rubber. In one example, the bladder is made of plastic. In one example, the bladder is made of metal.

In one embodiment, the Rigid Pressurized Container comprises a First Chamber and a Second Chamber;

wherein the First Bladder is contained within the First Chamber; and

wherein the Second Bladder is contained within the Second Chamber.

In one embodiment, the apparatus disclosed herein comprises a Third Composition. In one embodiment, the Third Composition is expelled into the Third Container. In one embodiment, the Third Composition is expelled into the ambient atmosphere. In one embodiment, the Third Composition activates the First Composition.

As used herein the term “activates” refers to reacting with a composition, such as the First Composition, thereby making the composition differently (e.g., more) reactive towards other molecules or chemical reactions.

In one embodiment, the Third Composition activates the Second Composition.

In one embodiment, the Third Composition neutralizes the First Composition.

As used herein the term “neutralizes” refers to lessening the chemical reactivity of a molecule. For example, neutralizing a composition, such as the First Composition, includes reacting with that composition, thereby rendering the composition less reactive towards other molecules or chemical reactions.

In one embodiment, the Third Composition neutralizes the Second Composition.

In one embodiment, the First Container is a different volume from the Second Container.

In one embodiment, each of the First Dispenser and the Second Dispenser is independently chosen from a spray nozzle, an atomizer, and a tube.

As used herein the term “spray nozzle” refers to a device that facilitates dispersion of liquid into a spray.

As used herein the term “atomizer” refers to a device, which forms a dispersion of fine solid particles or liquid droplets upon passing a material through the device.

In one embodiment, the apparatus is coated with an antibacterial.

As used herein the term “antibacterial” refers to a compound that either inhibits bacterial growth or kills bacteria.

In one embodiment, the Third Container is a toilet.

As used herein, the term “toilet” means a sanitation fixture used to collect or dispose of human feces or urine. Examples within the context of this disclosure include conventional toilet bowls, urinals, outhouses, and portable toilets.

In one embodiment, the toilet has a water holding tank. In one embodiment, the First Container is positioned inside the water holding tank.

As used herein, the term “water holding tank” means a tank which holds a cached volume of water for later use, wherein the tank may be filled at a rate that is independent of the rate at which the tank is emptied. In one embodiment, the term “water holding tank” includes a container which holds water along with devices and plumbing for a toilet.

In one embodiment, the toilet has a water holding tank and the First Container is positioned adjacent to the water holding tank.

As used herein, the term “positioned adjacent to the water holding tank” means the First Container is located next to the water holding tank but not inside of the water holding tank.

In one embodiment, the apparatus comprises a First Fill Level Indicator.

In one embodiment, the apparatus comprises a UV light, positioned to radiate the surrounding ambient atmosphere.

In one embodiment, the apparatus comprises a UV light, positioned to radiate the apparatus.

In one embodiment, the toilet has a toilet seat. In one embodiment, the toilet is equipped with a Sensor. In one embodiment, the Sensor triggers deployment of the First Composition.

As used herein, the term “First Fill Level Indicator” means a display means, which provides visible information about the amount of material present within one or more containers. In one embodiment, the “First Fill Level Indicator” is a light, such as an LED light. In one embodiment, the “First Fill Level Indicator” is a display screen.

As used herein, the term “toilet seat” means a hinged unit consisting of a seat (and optionally a lid), which is connected onto a toilet bowl for a toilet used in a sitting position.

As used herein, the term “Sensor” means a device that detects stimuli in its surrounding environment. Within the context of this disclosure the term “Sensor” includes infrared, electrical conductance, weight, and/or light sensors.

In one example, the Sensor activates the First Dispenser to deploy the First Composition. In one example, the Sensor detects the presence of a person sitting on a toilet seat of the apparatus. In one example, the apparatus deploys a First Composition into a toilet upon detecting the presence of a person sitting on a toilet seat.

As used herein, the term “trigger” means a stimulus which causes another event to occur. For example, the term “triggers deployment” means that an event (such as a Sensor detection) causes the First Composition to become deployed.

In one embodiment, the Sensor detects a water level in the toilet. In one embodiment, the Sensor detects gravitational force on the toilet set. In one embodiment, the Sensor detects electrical conductance on the toilet seat. In one embodiment, the Sensor triggers deployment of the Second Composition.

Although the present invention herein has been described with reference to various exemplary embodiments, it is to be understood that these embodiments are merely illustrative of the principles and applications of the present invention. Those having skill in the art would recognize that various modifications to the exemplary embodiments may be made, without departing from the scope of the invention.

Moreover, it should be understood that various features and/or characteristics of differing embodiments herein may be combined with one another. It is therefore to be understood that numerous modifications may be made to the illustrative embodiments and that other arrangements may be devised without departing from the scope of the invention.

Furthermore, other embodiments of the invention will be apparent to those skilled in the art from consideration of the specification and practice of the invention disclosed herein. It is intended that the specification and examples be considered as exemplary only, with a scope and spirit being indicated by the claims.

Finally, it is noted that, as used in this specification and the appended claims, the singular forms “a,” “an,” and “the,” include plural referents unless expressly and unequivocally limited to one referent, and vice versa. As used herein, the term “include” or “comprising” and its grammatical variants are intended to be non-limiting, such that recitation of an item or items is not to the exclusion of other like items that can be substituted or added to the recited item(s).

What is claimed is:

1. An apparatus comprising:
a First Container;
a Second Container;
a Third Container;
a First Composition;
a Second Composition;
a First Dispenser;
a Second Dispenser;
a surrounding ambient atmosphere;
wherein the First Dispenser expels the First Composition from within the First Container into the Third Container; and
wherein the Second Dispenser expels the Second Composition from within the Second Container into the surrounding ambient atmosphere.

2. (canceled)

3. The apparatus of claim 1, wherein the Third Container is open to the surrounding ambient atmosphere.

4. The apparatus of claim 1,
wherein the First Composition is held within the First Container;
wherein the Second Composition is held within the Second Container.

5. (canceled)

6. The apparatus of claim 1, wherein at least one of the First Container or the Second Container is a bag on valve container.

7. The apparatus of claim 1, wherein at least one of the First Container or the Second Container is a pump bottle.

8. A device comprising:
a Rigid Pressurized Container comprising a First Bladder and Second Bladder;
an Outlet;
wherein said First Bladder comprises a composition having a First Bladder Vapor Pressure;
wherein said Second Bladder comprises a composition having a Second Bladder Vapor Pressure;
wherein said Pressurized Container comprises a vapor pressure greater than one atmosphere of pressure; and
wherein said Pressurized Container comprises a vapor pressure greater than said First Bladder Vapor Pressure.

9. The apparatus of claim 8, wherein the Rigid Pressurized Container comprises a First Chamber and a Second Chamber;
wherein the First Bladder is contained within the First Chamber; and
wherein the Second Bladder is contained within the Second Chamber.

10. The apparatus of claim 1, comprising the device of claim 8.

11. The apparatus of claim 1, wherein the First Composition includes one or more ingredients chosen from an oil, a natural oil, a perfume, an oxidant, a soap, an acid, a base, a surfactant, an odor absorber, a cyclodextrin, or an antioxidant.

12. The apparatus of claim 1, wherein the Second Composition includes one or more ingredients chosen from an oil, a natural oil, a perfume, an oxidant, a soap, an acid, a base, a surfactant, an odor absorber, a cyclodextrin, or an antioxidant.

13. The apparatus of claim 1, comprising a Third Composition.

14. The apparatus of claim 1, wherein the Third Composition is expelled into the Third Container.

15. The apparatus of claim 13, wherein the Third Composition is expelled into the surrounding ambient atmosphere.

16. The apparatus of claim 14, wherein the Third Composition activates the First Composition or the Second Composition.

17. (canceled)

18. The apparatus of claim 14, wherein the Third Composition neutralizes the First Composition or the Second Composition.

19. (canceled)

20. (canceled)

21. The apparatus of claim 1, wherein each of the First Dispenser and the Second Dispenser is independently chosen from a spray nozzle, an atomizer, or a tube.

22. The apparatus of claim 1, wherein the apparatus is coated with an antibacterial.

23. The apparatus of claim 1 wherein the Third Container is a toilet.

24. The apparatus of claim 23, wherein the toilet has a water holding tank; and the First Container is positioned inside the water holding tank.

25. The apparatus of claim 23, wherein the toilet has a water holding tank; and the First Container is positioned adjacent to the water holding tank.

26. (canceled)

27. (canceled)

28. The apparatus of claim 23, wherein the toilet has a toilet seat;
wherein said toilet is equipped with a Sensor; and
wherein said Sensor triggers deployment of the First Composition or the Second Composition.

29. (canceled)

30. (canceled)

31. The apparatus of claim 28, wherein said Sensor detects electrical conductance on the said toilet seat.

32. (canceled)
